(19) United States
(12) Patent Application Publication
(10) Pub. No.: US 2026/0013862 A1
(43) Pub. Date: Jan. 15, 2026

(54) SYSTEM AND DEVICE FOR REDUCTION OF VARICOSE VEIN DIAMETER

(71) Applicant: BIOTRONIK AG, Buelach (CH)

(72) Inventors: Georg Nollert, Strasslach (DE); Aldo Jakob, Eglisau (CH); Sebastian Klaus, Embrach (CH)

(21) Appl. No.: 18/880,989

(22) PCT Filed: May 23, 2023
(86) PCT No.: PCT/EP2023/063814

§ 371 Date: Jan. 3, 2025

(30) Foreign Application Priority Data
Jul. 8, 2022 (EP) 22183851.9

(51) Int. Cl. A61B 17/12; A61B 17/00

(52) U.S. Cl. CPC A61B 17/12036; A61B 17/00234; A61B 17/12109; A61B 17/12131; A61B 2017/00238; A61B 2017/00292; A61B 2017/00477; A61B 2017/00862; A61B 2017/12054

(57) ABSTRACT

An implant delivery system for implanting an implant for reducing a luminal diameter of a vessel, including: an implant being configured to be anchored to an inner wall (W) of the vessel (V) and to contract so as to reduce the luminal diameter of the vessel (V), and a catheter including a catheter sheath and an expansion device for expanding and deploying the implant at an implantation side in the vessel (V).

### PRIORITY CLAIM

This application is a 35 U.S.C. 371 US National Phase and claims priority under 35 U.S.C. § 119, 35 U.S.C. 365 (b) and all applicable statutes and treaties from prior PCT Application PCT/EP2023/063814, which was filed May 23, 2023, which application claimed priority from EP Application Serial No. 22183851.9, which was filed Jul. 8 2022.

### FIELD OF THE INVENTION

A field of the invention is implant delivery systems and implants. The invention concerns a system and device configured for reducing varicose vein diameter.

### BACKGROUND

Varicose veins of the lower limbs have a very high incidence of up to 40% and cause 1-2% of all healthcare costs in western countries. Due to incompetent valves of the great saphenous vein (GSV) the venous blood pressure enlarges the saphenous vein and the veins draining into the saphenous vein. Blood flow is slowed down and may lead to deep vein thrombosis (DVT). Current invasive strategies mainly aim to destruct the GSV by various methods, leading to substantial costs and morbidity.

There are various symptomatic treatments and invasive therapies, but they are often expensive, inefficient and/or destructive with consecutive morbidity.

Venous insufficiency often also leads to edema. Symptomatic therapies to reduce edema include elevation of the legs, which reduces increased venous pressure, or manual lymphatic drainage to flush out the edema. Compression therapy with stockings or bandages are most frequently used.

The mechanism of action consists of compression of the vein and the associated reduction in diameter, which leads to faster blood flow and allows extended, non-functional venous valves to function adequately again. This lowers the venous pressure.

In the presence of varicosis, invasive therapies can be surgical removal (so-called stripping) or closure by radiofrequency ablation or using adhesives. In addition, the varicose veins can also be dissected.

Furthermore, symptomatic drug therapies exist in particular to avoid thrombosis such as Coumarin (a-benzopyrone), flavonoids (y-benzopyrone), horse chestnut extracts (saponosides) as well as acetylsalicylic acid and pentoxifylline (see for example: Beebe-Dimmer J L, Pfeifer J R, Engle J S, Schottenfeld D. The epidemiology of chronic venous insufficiency and varicose veins. Ann Epidemiol. 2005 March; 15 (3): 175-84).

However, all known non-invasive therapies address the disease merely symptomatically. Furthermore, invasive endovascular therapies are often destructive. Moreover, usually, for using thermal devices or surgery, tumescent anesthesia is needed with associated infrastructure (sterile environment and operating room). Also, when removing varicose veins, they cannot be used later on e.g. for bypass surgery. Generally, procedure times are rather long, sterile operation theaters are needed and patient after-care can be relatively long-lasting. Further, the required imaging modalities can often be complex.

### SUMMARY OF THE INVENTION

Preferred embodiments provide a medical implant that can be implanted in a minimally invasive fashion and that ideally preserves veins and venous valves, wherein the implant is adapted to reduce the venous diameter and consequently to accelerate the flow in the vein, and to enable sufficient venous valve closure and thereby lowering the venous pressure.

A preferred implant delivery system for implanting an implant for reducing a luminal diameter of a human or animal vessel, particularly vein, includes:
an implant (e.g. spring clip) being configured to be anchored to an inner wall of the vessel via a first end section and an opposing second end section, wherein the implant includes a connecting portion configured to connect the first and second end section, wherein the implant is configured to assume a first state allowing the first and the second end section to be anchored to the inner wall and a second state for reducing the luminal diameter, wherein the first end section and the second end section are further apart from one another in the first state than in the second state, and
a catheter for delivering the implant to an implantation site in the vessel, the catheter being further configured to arrange the implant in the first state for anchoring the implant to the inner wall, and optionally thereafter in the second state to reduce the luminal diameter of the vessel.

### BRIEF DESCRIPTION OF THE DRAWINGS

In the following, embodiments of the present invention as well as further features and advantages of the present invention shall be described with reference to the Figures, wherein

FIG. 1 shows an embodiment of an implant according to the present invention together with an expansion device including legs for expanding and anchoring the implant,

FIG. 2 shows a cross-sectional schematic view of the anchoring process of implant using the expansion device shown in FIG. 1,

FIG. 3 shows the reduction of the inner diameter of the vein due to the implant according to the present invention,

FIG. 4 shows a schematic cross-sectional view of an embodiment of an implant delivery system according to the present invention,

FIG. 5 shows a variant of an elongated guiding member of the embodiment shown in FIG. 4,

FIG. 6 shows a further embodiment of an implant delivery system according to the present invention,

FIG. 7A-C show a further embodiment of an implant delivery system according to the present invention,

FIG. 8A/B show a further embodiment of an implant delivery system according to the present invention including a mesh structure mechanism,

FIG. 9 shows an embodiment of an implant according to the present invention together with an expansion device including legs for anchoring implant parts, wherein here the implants include two connecting portions being initially disconnected,

FIG. 10 shows the embodiment of FIG. 9 with the parts of the connecting portions being connected to reduce the luminal diameter of the vein,

FIG. 11A-G show different embodiments for connecting the parts of the connecting portion of the implant, and

FIG. 12 shows an embodiment of an implant according to the present invention utilizing several end sections for anchoring the implant on either side of the inner wall.

### DETAILED DESCRIPTION OF THE PREFERRED EMBODIMENTS

A preferred implant delivery device can be a minimally invasive catheter/endoscopic device through which the implant can be anchored to the inner wall of the dilated venous vessel. Particularly, after implantation, the implant may contract and thereby reduces the luminal diameter of the vessel. Further, particularly, the present invention thus allows reconstructing the valves by reducing the luminal cross section of the vessel, particularly by using an implant (or several such implants) in form of a tightening spring clip. These implants/clips preferably perforate the venous wall at two sites and pull them together thereby reducing the diameter of the vessel and making the venous valves competent again.

Particularly, regarding all embodiments described herein, the vessel can be a vein, particularly a varicose vein. In the following, the invention is described in conjunction with veins, but the implant can generally be applied also to other blood vessels.

Furthermore, in an embodiment of the invention, the first end section of the implant includes a first fixation arm and a first tip for anchoring the first end section to the inner wall of the vessel, and wherein the second end section includes a second fixation arm and a second tip for anchoring the second end section to the inner wall of the vessel. The respective tip can be configured for piercing the wall of the vein, wherein particularly the respective fixation arm can be configured to anchor the implant to the wall of the vein. Particularly, the respective tip can be formed at an end of the respective fixation arm, e.g. at a location where the respective fixation arm is connected to a remainder of the corresponding (first or second) end section.

Further, according to a preferred embodiment of the present invention, the catheter includes a catheter sheath and an expansion device slidably arranged in a lumen of the catheter sheath, wherein the expansion device is configured to move the first end section and the second end section apart from one another to arrange the implant in the first state for anchoring the implant to the inner wall.

Furthermore, the first end section includes a first connection arm configured to releasably engage with the expansion device, and wherein the second end section includes a second connection arm configured to releasably engage with the expansion device, too.

According to yet another embodiment of the invention, the expansion device includes a first and a second leg (for example made from a nickel titanium alloy such as NiTinol) configured to spread apart (particularly elastically) from one another to transform the implant to assume its first state and anchor the first and the second end section of the implant in the inner wall of the vein, wherein particularly the first end section is configured to releasably engage with the first leg and the second end section is configured to releasably engage with the second leg of the expansion device. Particularly, due to this engagement the expansion device can transfer a force to the implant allowing the implant to be anchored to the inner wall of the vein via said first and second end sections.

According to a further embodiment of the invention, the expansion device includes a third leg which spreads apart from the first and the second leg for positioning the first and the second leg close to the inner wall of the vein. The third leg may preferably be made of a self-expanding material such NiTinol. Particularly the three legs are spaced apart from one another along a circumferential direction of the expansion device by 120°.

Particularly, according to an embodiment of the present invention, when the implant is being deployed in the vessel, the first and the second connection arm are configured to disengage from the first and the second leg of the expansion device, particularly so as to move into a position in-plane with the contracting member, in particular for providing close contact to the wall of the vein and particularly also for becoming covered by venous tissue to reduce contact of the connection arms with blood, which could lead to thrombus formation.

Furthermore, according to a preferred embodiment of the present invention, the connecting portion of the implant connects the first end section to the second end section, particularly integrally, and is elastic, so that the connection portion is expanded when the implant is arranged in the first state and a restoring force is generated that forces the implant to assume the second state, i.e. to pull the first and the second end sections towards one another, when the implant is released from the expansion device (e.g. when the connection arms disengage from the legs of the expansion device in an embodiment).

Furthermore, according to a preferred embodiment of the invention, the contracting member includes a meandering shape. Furthermore, particularly, the contracting member, or even the whole implant, can be formed out of a nickel titanium alloy such as NiTinol.

According to yet another preferred embodiment, the connecting portion includes a first part connected to the first end section and a second part connected to the second end section of the implant, wherein in the first state of the implant the first part and the second part are disconnected from one another, and wherein in the second state, the first part and the second part are connected to one another so as to form a continuous connection (connecting portion) between the two opposing first and second end sections to thereby reduce said luminal diameter of the vein (i.e. due to said connection, the first and second end section are closer to one another in the second state of the implant when compared to the first state of the implant).

Furthermore, according to a preferred embodiment of the present invention, the first and the second part are configured to be connected to one another by at least one snap-in connection formed by the first and the second part in the second state of the implant, i.e., the first and the second part engage with one another thereby forming said snap-in connection. According to an alternative preferred embodiment, the first and the second part of the connecting portion are configured to be connected by a magnetic force in the second state of the implant. For this, the first and the second part can be formed as permanent magnets or can each include a permanent magnet. According to yet another preferred alternative embodiment, the first and the second part of the connecting portion of the implant are configured to be connected by engaging with one another, wherein the first part preferably includes at least one loop engaging with at least one hook included by the second part of the connecting portion in the second state of the implant.

Further, according to an embodiment of the present invention, the catheter includes a movable sheath configured to be advanced with respect to the expansion device or with respect to the outer sheath so as to move the first leg and the second leg towards one another and to thereby connect the first part to the second part of the connecting portion in the second state of the implant.

Furthermore, according to a preferred embodiment of the present invention, the implant is configured to be anchored to the inner wall of the vessel via a plurality of first end sections and a plurality of opposing second end sections. Preferably, the first end sections are rigidly connected to one another, particularly by an elongated first connecting element, and wherein the second end sections are preferably rigidly connected to one another, too, particularly by an elongated second connecting element. Furthermore, preferably, the connecting portion is configured to connect the first and second end sections, wherein the implant is configured to assume a first state allowing the first and the second end sections to be anchored to the inner wall and a second state for reducing the luminal diameter of the vein, wherein the first end sections are further apart from the second end sections in the first state than in the second state so as to allow anchoring of the first and second end sections in the inner wall of the vein. Further, preferably, the connecting portion is an elongated flexible member (such as a wire or suture) connecting the plurality of first end sections to the plurality of second end sections such that a pulling force applied to the connecting portion pulls the first end sections and the second end sections towards one another so as to arrange the implant in the second state. Particularly, due to the connecting elements that can extend longitudinally along the vein/inner wall and may extend essentially parallel to one another, the first and second on one side and the second end section on the other side are not pulled towards one another in the axial direction (i.e. along the axial extension direction of the vein), but in a transversal direction which leads to the desired reduction of the luminal diameter of the vein (second state of the implant).

According to a further embodiment of the present invention, the implant delivery system further includes an elongated guiding member arranged in the lumen of the catheter sheath, the guiding member including a first and a second sidewall opposing one another and being connected by a connecting member. Particularly, in an embodiment, the guiding member is a U-shaped profile. Preferably, the first side wall includes an elongated first slot and the second side wall includes an elongated second slot, wherein the first and the second leg of the expansion device are configured to be slidably arranged in the lumen of the catheter sheath between the first sidewall and the second sidewall of the guiding member, and wherein the implant is configured to be arranged (particularly in a bent shape, particularly in a semi-circular shape) in the lumen of the catheter sheath so that the first connection arm engages into the first slot and into a first opening of the first leg of the expansion device and the second connection arm engages into the second slot and into a second opening of the second leg of the expansion device.

According to a further embodiment, the first and the second leg are configured to spread apart from one another when the first and the second legs are moved out of the lumen of the catheter sheath through an opening of the catheter sheath at a distal end of the catheter sheath to anchor the first and the second fixation arm in the inner wall of the vessel and to expand the elastic connecting portion of the implant. Due to expansion of the connecting portion, two opposing vessel wall portions (i.e. where the fixation arms are anchored) are pulled towards one another to achieve the reduction of the inner diameter of the vein (second state of the implant). Alternatively, as described above, the wall portions are pulled toward one another when the two parts of the initially disconnected connecting portion get connected in the second state of the implant.

Furthermore, according to an embodiment, the first connection arm is configured to move out of the first opening of the first leg and the second connection arm is configured to move out of the second opening of the second leg of the expansion device when the expansion device is retracted into the lumen of the catheter sheath.

According to a further embodiment of the present invention, the implant delivery system includes further implants (each further implant being in particular configured as the implant according to the present invention), wherein each further implant is configured to be anchored to the inner wall of the vein and to have its connecting portion contracted or the parts of its connecting portion connected (particularly after anchoring) so as to reduce the luminal (i.e. inner) diameter of the vein, wherein each further implant includes a first and a second end section opposing one another and configured to anchor the further implant to the inner wall of the vein, and a connecting portion (e. g. elastic connecting portion) for connecting the first and the second end sections to one another, the connecting portion being configured to pull the first and the second end sections towards one another to contract the further implant (and to thereby reduce the luminal diameter of the vein) or to have its (initially) disconnected parts connected as described above.

Particularly, the first end section of the respective further implant in turn includes a first fixation arm and a tip for anchoring the first end section to the inner wall of the vessel, and wherein the second end section includes a second fixation arm and a tip for anchoring the second end section to the inner wall of the vessel (see also above), and wherein preferably the first end section includes a first connection arm configured to releasably engage with the expansion device, and wherein the second end section includes a second connection arm configured to releasably engage with the expansion device, namely with said openings of the first and second legs. Particularly, the further implants are configured to be arranged behind said implant one after the other so that the first connection arms of the further implants run through the first slot of the first sidewall of the guiding member and the second connection arms of the further implants run through the second slot of the second sidewall of the guiding member, wherein the first and the second legs run between the first and second connection arms of the further implants. The further implants can each be designed as the leading implant described above.

According to a preferred embodiment, the first leg of the expansion device is also configured to engage with the first connection arm of a further implant forming the leading implant after deployment of said (formerly foremost) implant when being retracted into the lumen of the catheter sheath, and the second leg of the expansion device is configured to engage with the second connection arm of the further (now leading) implant when being retracted into the lumen of the catheter sheath.

According to yet another embodiment, the expansion device is formed by an expandable balloon including a portion configured to carry said implant, wherein the catheter sheath is retractable to remove said catheter sheath from said portion of the balloon to allow expansion of said portion of the balloon to expand and anchor the implant to the inner wall of the vein. The implant can be designed as described above.

Particularly, the delivery system includes further implants, each further implant being arranged on a respective portion of said balloon, wherein the catheter sheath is retractable to remove said catheter sheath from the respective portion of the balloon to allow expansion of the respective portion of the balloon to expand and anchor the respective further implant thereon to the inner wall of the vein.

According to yet another embodiment, the expansion device includes two spreading elements actuated by a threaded rod, and two profiles, each profile configured to travel on top of one of the spreading elements to expand and anchor said implant to the inner wall of the vein, wherein particularly, the delivery system includes further implants each implant being arranged on said profiles to subsequently expand and anchor the respective further implant to the inner wall of the vein. In an alternative embodiment the expansion device includes a mesh structure based mechanism capable of expansion, preferably actuated by a threaded rod or a slidable tube.

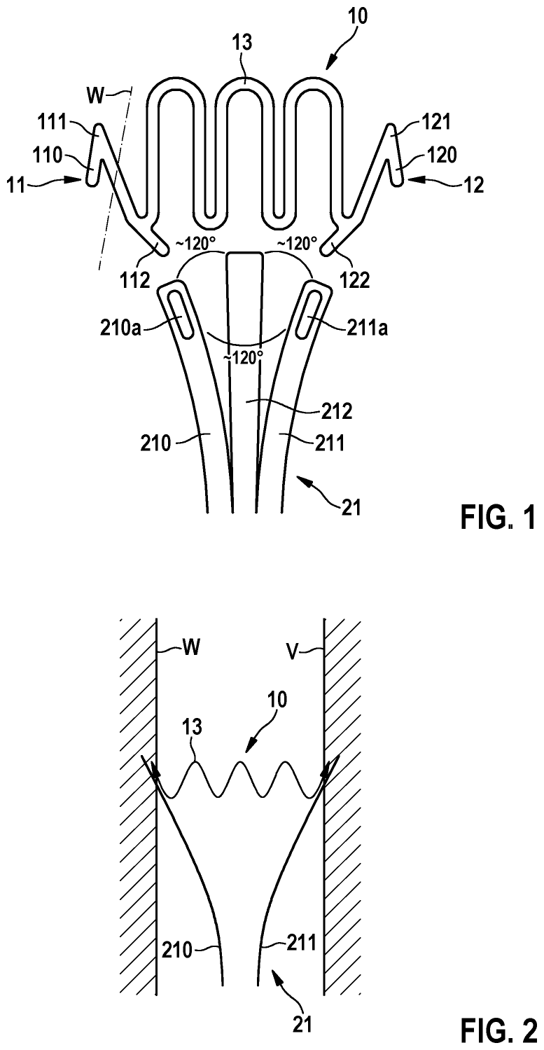

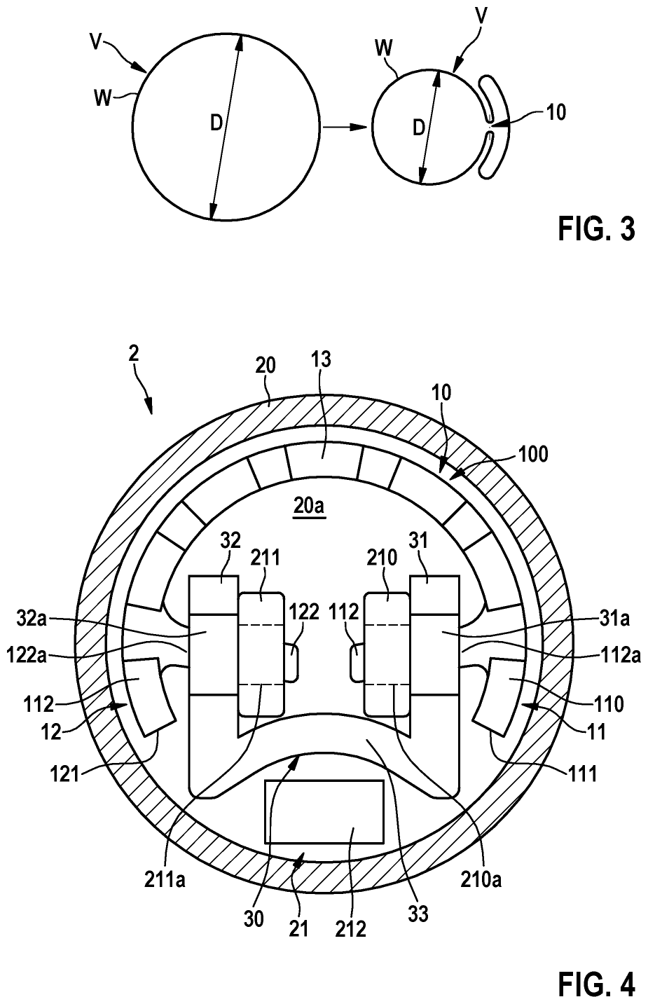

The present invention relates to an implant delivery system 1 for implanting an implant 10 for reducing a luminal (i.e. inner) diameter D of a vessel (cf. FIG. 3), including: an implant 10 being configured to be anchored to an inner wall W of the vessel V, particularly a vein V, and to undergo a contraction, so as to reduce the luminal diameter D of the vessel/vein V. The system 1 further includes a catheter 2 including a catheter sheath 20 and an expansion device 21 for deploying the implant 10. Embodiments of such a delivery system 1 are e.g. shown in FIGS. 1, 4, 6, 7, 8, 9 and 10.

The invention thus allows to place tightening implants 10 (e.g. in form of spring-clips), for example made from a nickel-titanium-alloy such as NiTinol (NiTi), in the affected vein sections (see for instance FIG. 2).

Preferably, as indicated in FIG. 1, each implant 10 (e.g. clip) can include a connecting portion 13 such as an elastic connecting portion 13, i.e. spring element, that can form a spring-like center-section 13 of the respective implant 10, which connecting portion 13 connects two opposing end sections 11, 12 of the respective implant 10, which end sections 11, 12 are adapted for entering the vessel wall W and fixating the implant 10 to the vessel wall W, wherein an entering part of the respective end section 11, 12 has a pointed tip 111, 121 as shown e.g. in FIG. 1. The connecting portion 13 is configured to pull the first and the second end sections 11, 12 towards one another to contract the implant 10. The connecting portion 13 can include a meandering shape.

Further, as indicated in FIG. 1, the first end section 11 of the implant 10 can include a first fixation arm 110 extending from the first tip 111 for anchoring the first end section 11 to the inner wall W of the vessel V. Likewise, the second end section 12 can includes a second fixation arm 120 extending from the second tip 121 for anchoring the second end section 12 to the inner wall W of the vessel V. The respective fixation arm 110, 120 and the associated tip 111, 121 can form a hook structure.

According to an embodiment, the implant 10 can be deployed in the vein V by using an expansion device 21 that includes a first and a second leg 210, 211, for example made from NiTinol (NiTi), and configured to spread elastically outward. By the two leg-like elements 210, 211 the two anchoring end sections 11, 12 of the implant/clip are guided into the wall W of the vein V as illustrated in FIGS. 1 and 2, wherein a connection arm 112, 122 of the respective end section 11, 12 is engaged with an associated leg 210, 211, wherein particularly the respective connection arm 112, 122 can engage with an opening 210a, 211a provided in the respective leg 210, 211. At the same time, while guiding the tips 111, 121 into the wall W, the legs 210, 211, due to spreading outwards, expand the elastic connecting portion 13. The expansion force particularly comes from the legs 210, 211 that can be pre-shaped to assume the configuration with legs 210, 211 spreading outwards.

As shown in FIG. 1, the expansion device 21 can include a third leg 212 that provides positioning to get the two other legs 210, 211 and the implant 10 close to the wall W of the vessel/vein V. For this the spread configuration of the legs 210, 211, 212 preferably includes that the legs 210, 211, 212 are spaced apart in a circumferential direction of the expansion device 21 of about 120° as indicated in FIG. 1 which results in a reduction in vein diameter of about 30%.

During deployment, the first and second tips 111, 121 of the anchoring end sections 11, 12 enter the vein wall W together with the respective fixation arm 110, 120. After fixation in the wall W the legs 210, 211, 212 are drawn backwards and the connection of implant 10 and legs 210, 211 is released, particularly due to the fact that the connections arms 112, 122 move out of the respective opening 210a, 211a.

Since no expansion force is acting on the implant 10 anymore, its spring-like connecting portion 13 contracts and tightens the vein wall W thereby reducing the luminal diameter D as shown in FIG. 3 and restoring valve function.

When being deployed in the vessel V, the connection arms 112, 122 preferably return elastically into a position in-plane with the implant's 10 center section, i.e., connecting portion 13, providing close wall contact, and are covered by the venous tissue to avoid the foreign material contact with blood leading potentially to thrombus formation.

Due to the arrangement of the legs 210, 211 and the connections arms 112, 122 and force directions of the implant 10 the implant 10 is continuously kept under spring force in order to not jumping off the legs 210, 211 during deployment.

Furthermore, the implant 10 and the legs 210, 211, 212 are preferably brought to the target location by using a minimal-invasive device like a catheter 2. An embodiment of a corresponding implant delivery system based on a catheter 2 is shown in FIG. 4 in a cross-sectional view perpendicular to a longitudinal axis of the catheter 2.

As indicated in FIG. 4 the implant 10 can be placed inside a lumen 20a of a catheter sheath 20, particularly in a bent (e.g. semi-circular) configuration. The connection arms 112, 122 of the implant 10 are running through axial slots 31a, 32a in the side walls 31, 32 of a guiding member 30 that can be shaped as a U-profile and includes two opposing side walls 31, 32 connected by a connecting member 33 forming a bottom of the guiding member 30. Particularly, the guiding member 30 is placed in the center of the lumen 20a of the catheter sheath 20. At the same time the connection arms 112, 122 are placed inside the openings 210a, 211a of the legs 210, 211.

Due to the pre-shaped setting, the legs 210, 211 are providing permanent outward force pressing against the inner surfaces of the side walls 31, 32 of the guiding member 30. Further, the connection arms 112, 122 can have a necking area 112a, 122a where they run in the slots 31a, 32a to improve engagement with the slots 31a, 32a to ensure that the connection arms 112, 122 cannot jump out of the slots 31a, 32a. If so, loading the implant(s) 10 onto the guiding member 30 (e.g. U-profile) can be made from one face side of the guiding member 30 during assembly. For stability reasons the guiding member (e.g. U-profile) can have a separate top wall (not shown) being assembled onto it after loading of implant(s) 10 and legs 210, 211. The top wall would thus be arranged so as to face the bottom 33.

To be able to deliver multiple implants 10 with one catheter 2, the implants 10 are placed inside the catheter lumen 20a axially behind each other (e.g. like in a stapler). The connection arms 112, 122 of all implants 10 run through the slots 31a, 32a in the guiding member 30. Just the implant 10 (e.g. clip) most distal has its connection arms 112, 122 placed in the connection openings 210a, 211a of the legs 210, 211. For the other implants, the two legs 210, 211 run between tips of the connection arms 112, 122. Therefore, a gap at the center of the guiding member 30 (e.g. U-profile) is preferably wide enough to allow the two legs 210, 211 to travel through.

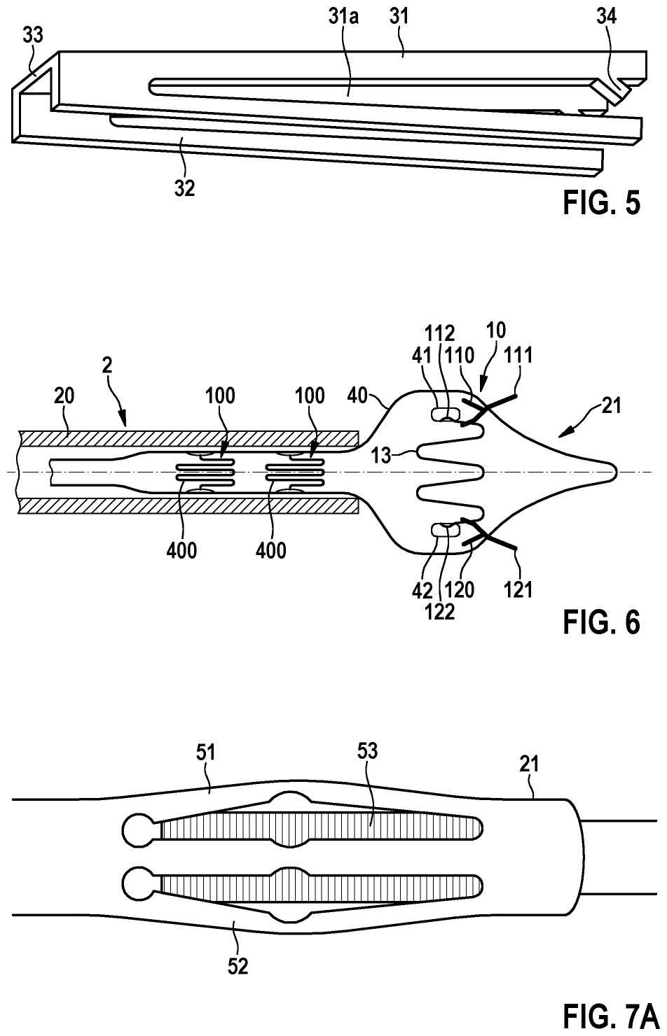

Furthermore, as indicated in FIG. 5, which shows a lateral view of the guiding member 30, the slots 31a, 32a formed in the side walls 31, 32 of the guiding member 30 can include a contour shape allowing for a ratchet-like mechanism with the implant(s). For this, the guiding member 30 may include a protrusion 34 at each distal end of the respective slot 31a, 32a, thus narrowing the respective slot 31a, 32a locally.

Particularly, a delivery of several implants (e.g. clips) 10 as shown in FIG. 1 could be conducted as follows using e.g. the catheter 2 shown in FIGS. 4 and 5: When pushing the legs 210, 212 forward the implant 10 most distal is pushed forward at the same time since the connection arms 112, 122 and the leg openings 210a, 211a are in engagement.

When the implant 10 leaves the distal catheter opening, the legs 210, 211, 212 are spreading elastically outward and the delivery into the venous wall takes place as described above. After fixation of this implant 10 in the wall W the legs 210, 211, 212 are pulled back into the lumen 20a of the catheter sheath 20 travelling through the guiding member's 30 center until the openings 210a, 211a find the connection arms 112, 122 of the further (i.e. second) implant 100 and snap in. Now carrying the further implant 100, the legs 210, 211 are pushed forward again to deliver that implant and so on. According to an embodiment, the number of implants per catheter can be in the range from 4 implants to 6 implants. Note, that FIG. 4 shows a view from the distal and onto the shown cross-section. Thus, the leading implant 10 is shown, wherein the further implants 100 are arranged behind this implant and are thus not visible.

FIG. 6 shows an alternative embodiment of the implant delivery system 1 according to the present invention, wherein instead of using the elastic forces stored in the pre-shaped (e.g. NiTinol) guiding legs 210, 211, 212 to expand the spring-like contracting member 13 of the implant 10, expansion of the implant 10, which can be formed as described in conjunction with FIGS. 1 and 2, can be provided by an alternative expansion device 21. Here, the expansion device 21 can include a dilatation balloon 21 carrying at least one or several axially pre-mounted implants 10, 100 and a catheter 2 providing a retractable catheter sheath 20 just allowing the most distal implant, here implant 10, to be expanded by the balloon 21 and delivered. Particularly, the respective implant 10, 100 is mounted to a portion 40, 400 of the balloon 21, wherein here the connection arms 112, 122 of the respective implant 10, 100 can engage with features provided on the respective portion 40 of the balloon (such as e.g. openings 41, 42) as shown with respect to the implant 10 currently being deployed in FIG. 6.

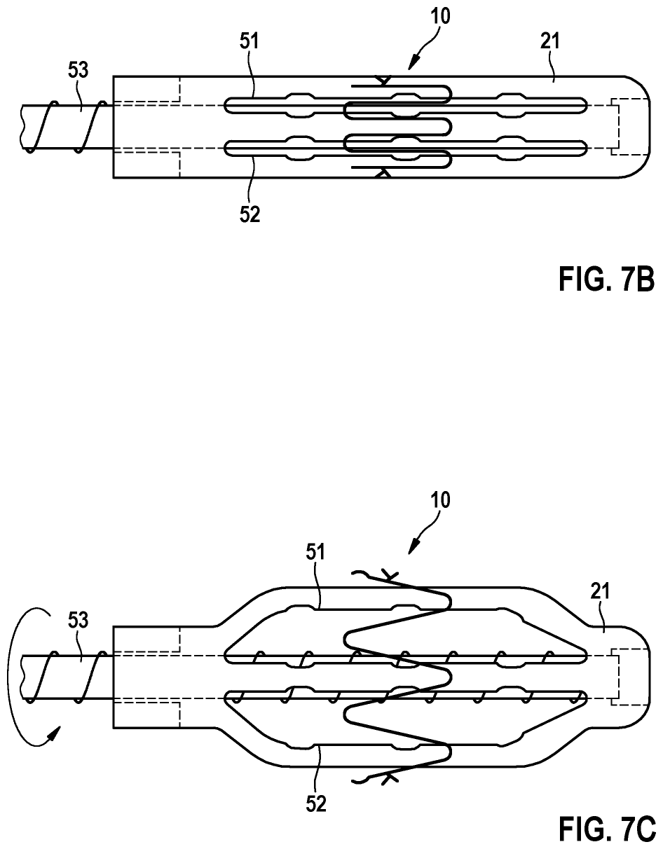

Furthermore, FIG. 7A-C show yet another embodiment of an implant delivery system 1 according to the present invention.

Here, expansion of the respective implant 10 can be provided by an expansion device 21 that is actuated by e. g. a threaded rod 53.

Particularly, in the embodiment shown in FIG. 7B/C, two profiles can be provided, each profile configured to travel on top of one of the spreading elements 51, 52 to expand and anchor said implant 10 to the inner wall W of the vessel V, wherein particularly, the delivery system 1 includes further implants, each further implant being arranged on said profiles to subsequently expand and anchor the respective further implant to the inner wall W of the vessel V. The implants would then be mounted on said profiles axially traveling on top of two spreading elements 51, 52 subsequently delivering the implants at the outer-most position of the spreading elements 51, 52.

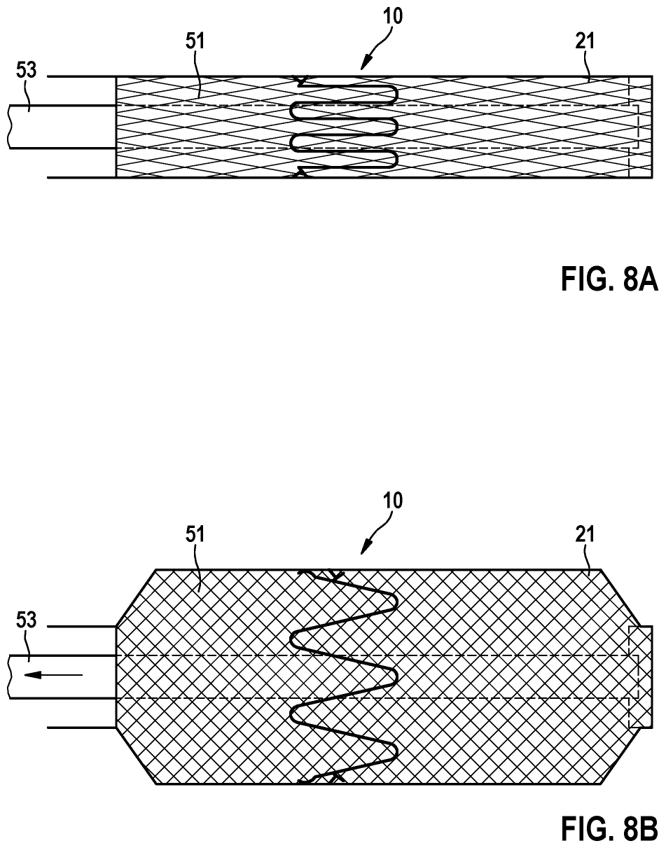

Alternatively, and as shown in FIG. 8A/B, the expansion device 21 is provided in the form of a mesh which could be made of a shape memory alloy such as a nickel titanium alloy (e.g. NiTinol). The spreading element 51 in this embodiment is a mesh or a braid with its struts or wires. It is thereby also conceivable that expansion device 21 is actuated by a threaded rod 53 or that the mesh is erected by a forward sliding of expansion device 21 on rod 53 or by withdrawal of rod 53 versus the expansion device 21.

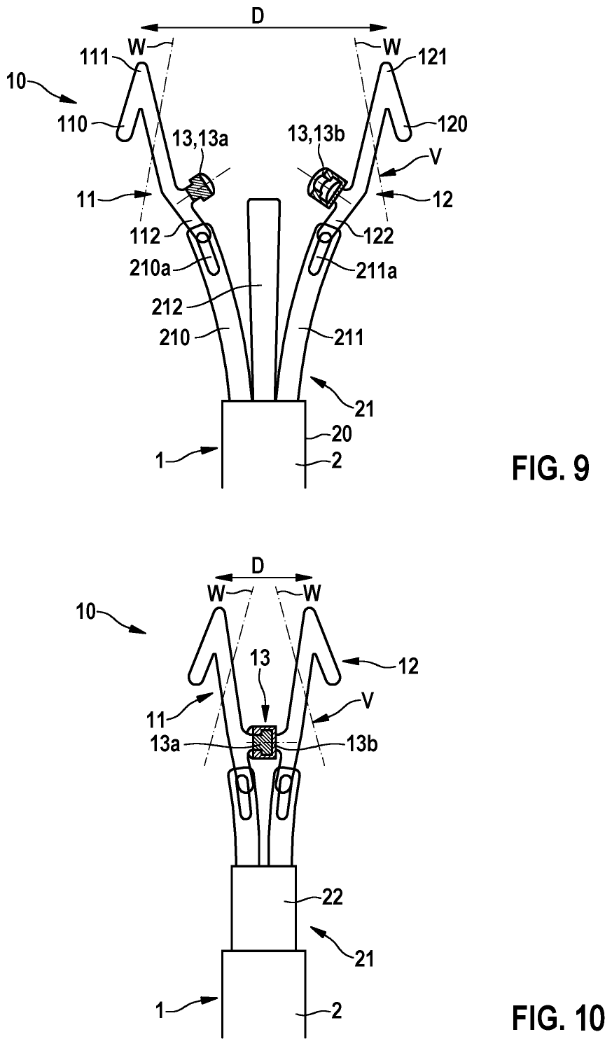

FIG. 9 shows in conjunction with FIG. 10 a further embodiment of an implant delivery system according to the present invention, wherein here, in contrast to the embodiment shown in FIG. 1, the connecting portion 13 does not initially connect the anchoring end sections 11, 12 to one another, but includes a first part 13a connected to the first end section 11 and a second part 13b connected to the second end section 12 of the implant 10, wherein in the first state of the implant 10 the first part 13a and the second part 13b are disconnected and separated from one another as e.g. shown in FIG. 8 which allows the first and second end sections 11, 12 to be anchored to the inner wall W of the vein by moving the end sections 11, 12 outwards when being in engagement with the legs 210, 211 of expansion device 21. Particularly, anchoring can be achieved by piercing the wall W by the tips 111, 121 of the end sections 11, 12 and anchoring the end sections 11, 12 by their fixation arms 110, 120. The parts 13a, 13b of the connecting portion 13 can be arranged side-by-side when still residing inside the sheath 20 of catheter 2 so that they are not connected to one another. However, after having anchored the end sections 11, 12 of the implant 10 as shown in FIG. 8, a movable sheath 22 of the catheter 2 can be advanced as shown in FIG. 9, which causes the legs 210, 211 to approach one another in a direction transverse to their respective longitudinal axis so as to let the parts 13a, 13b engage with one another and form a continuous connecting portion 13 connecting the end sections 11, 12 and holding them close to one another which reduces the luminal diameter D of the vein.

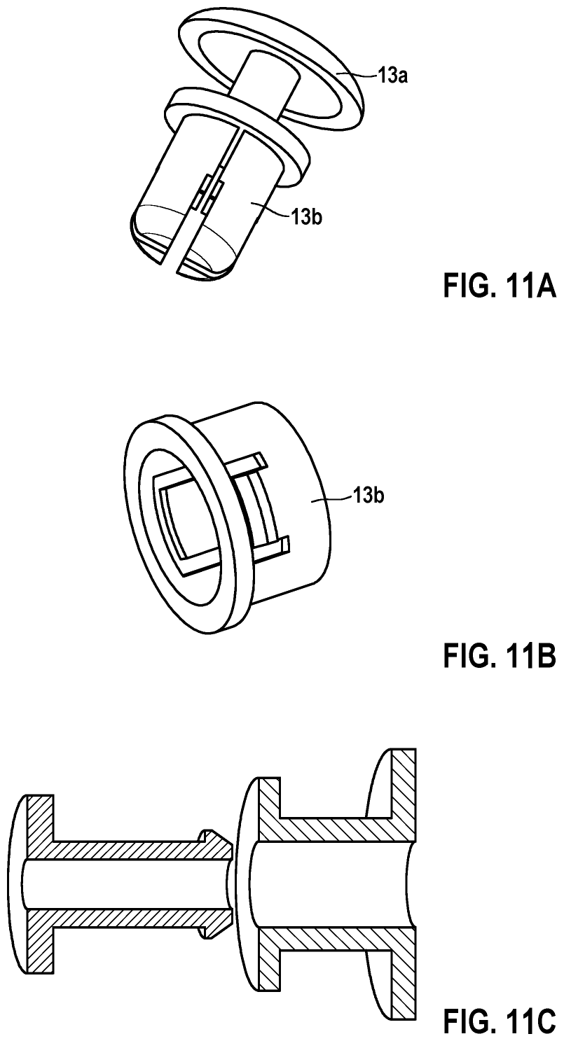

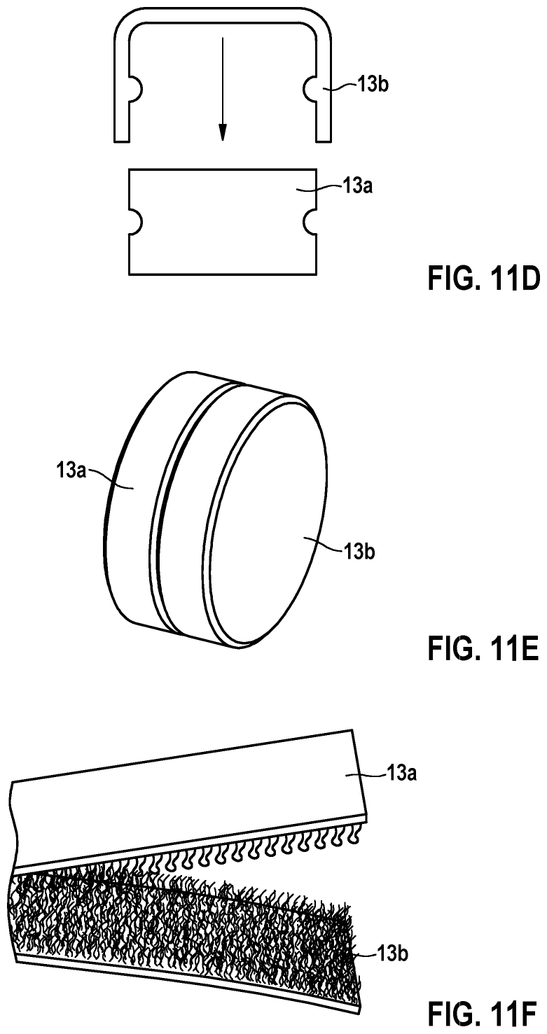

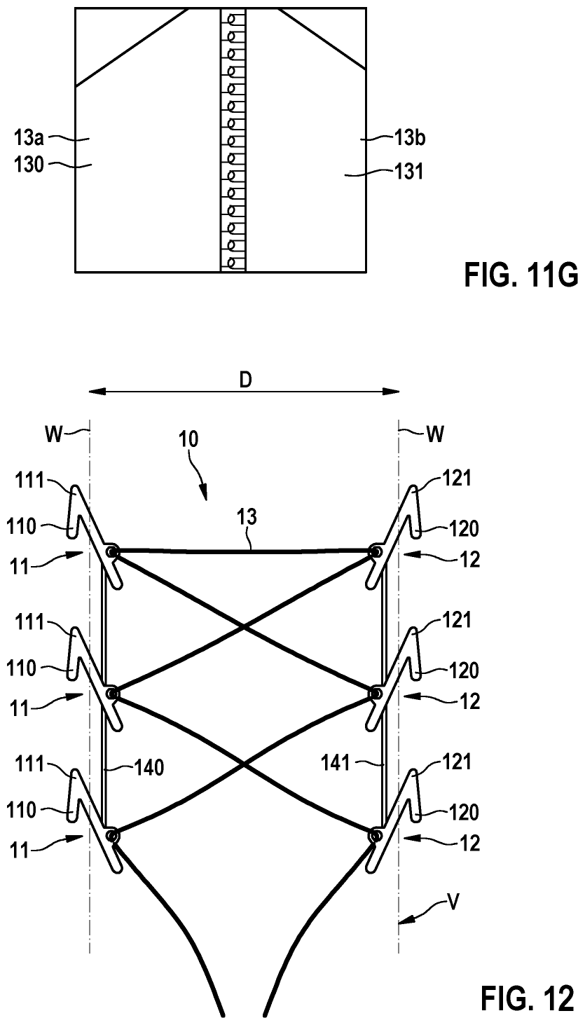

FIG. 11A to 11G illustrate different variants of embodiments of connections between the parts 13a, 13b of the connecting portion 13 that can be used. Particularly, as shown in FIG. 11A to 11D, the connection can be a snap-fit connection wherein one of the parts 13a can engage with the other part 13b to hold the two parts 13a, 13b together. Alternatively, magnetic parts 13a, 13b may be employed as shown in FIG. 11E so as to hold the part 13a, 13b together by utilizing a magnetic attractive force between the part 13a, 13b. Furthermore, the parts 13a, 13b can be configured to achieve multiple snap-fit connections as indicated in FIG. 11F such as implemented in Velcro or may utilize one or several loops 130 engaging with a one or several hooks 131 as shown in FIG. 11G.

Furthermore, FIG. 12 shows yet another embodiment of an implant 10 of a delivery system according to the present invention, wherein the implant 10 is configured to be anchored to the inner wall W of the vessel V via a plurality of first end sections 11 and a plurality of opposing second end sections 12, wherein the first end sections 11 are rigidly connected to one another by an elongated first connecting element 140, while the second end sections 12 are rigidly connected to one another by an elongated second connecting element 141 in an analogous fashion. Here, the connecting portion 13 is a flexible elongated member (e.g. a wire, rope or suture) that extends back and forth between the first and second end sections 11, 12 so that a pulling force applied to one or two ends of the connecting portion 13 pulls the first end sections 11 and the second end sections 12 towards one another so as to reduce the luminal diameter D. Due to the connecting elements 140, 141 that extend in the axial direction of the vein V along the inner wall W, the first and second end sections 11, 12 are merely pulled towards one another transverse to the connecting elements 140, 141, but not in said axial direction. Also, regarding the embodiment shown in FIG. 12, the implant 10 can be advanced out of a catheter sheath, wherein the two opposing structures including the first end sections 11 connected to the first connecting element 140 on one side and the second end sections 12 connected to the second connecting elements 141 on the other side can be moved away from one another to anchor the end sections 11, 12 in the inner wall W (first state) by an appropriate expansion device. Thereafter, by pulling on the connecting portion 13, the implant 10 can be transitioned into the second state leading to a reduction in luminal diameter D.

The invention presented has several clinical advantages. Particularly, the invention provides a minimal invasive, non-destructive approach with preservation of the venous vessels (the saphenous vein is reconstructed and preserved for further use, e.g. bypass surgery). Furthermore, the invention allows to avoid certain complications (e.g. allergies), is expected to include a short intervention time (<30 min), wherein particularly patients can walk immediately after the procedure. Further, only a venous puncture is needed; no sterile operating room is needed. The procedure can be done office based with ultrasound (US) as the only imaging modality. On the technical side, the invention can be implemented in a purely mechanical fashion and thus does not need a further energy source or active medical device; no console device is required.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. An implant delivery system for implanting an implant for reducing a luminal diameter (D) of a vessel (V), comprising:
an implant configured to be anchored to an inner wall (W) of the vessel (V) via a first end section and an opposing second end section, wherein the implant comprises a connecting portion configured to connect the first and second end section, wherein the implant is configured to assume a first state allowing the first and the second end section to be anchored to the inner wall (W) and a second state to reduce the luminal diameter, wherein the first end section and the second end section are further apart from one another in the first state than in the second state, and
a catheter configured to deliver the implant to an implantation site in the vessel (V), the catheter further being configured to arrange the implant in the first state to anchor the implant to the inner wall, and to arrange the implant in the second state to reduce the luminal diameter (D) of the vessel (V).

2. The implant delivery system according to claim 1, wherein the first end section comprises a first fixation arm and a first tip configured to anchor the first end section to the inner wall (W) of the vessel (V), and wherein the second end section comprises a second fixation arm and a second tip configured to anchor the second end section to the inner wall (W) of the vessel (V).

3. The implant delivery system according to claim 1, wherein the catheter comprises a catheter sheath and an expansion device slidably arranged in a lumen of the catheter sheath, wherein the expansion device is configured to move the first end section and the second end section apart from one another to arrange the implant in the first state.

4. The implant delivery system according to claim 3, wherein the first end section of the implant comprises a first connection arm configured to releasably engage with the expansion device, and wherein the second end section comprises a second connection arm configured to releasably engage with the expansion device.

5. The implant delivery system according claim 3, wherein the expansion device comprises a first and a second leg configured to spread apart from one another to arrange the implant in the first state and anchor the first and the second end section of the implant in the inner wall (W) of the vessel (V), wherein the first end section is configured to releasably engage with the first leg and the second end section is configured to releasably engage with the second leg of the expansion device.

6. The implant delivery system according to claim 5, wherein the expansion device comprises a third leg configured to position the first and the second leg close to the inner wall (W) of the vessel (V).

7. The implant delivery system according to claim 5, wherein when the implant is being deployed in the vessel (V), the first and the second connection arms are configured to disengage from the first and the second legs.

8. The implant delivery system according to claim 3, wherein the connecting portion connects the first end section to the second end section and is elastic, so that the connection portion is expanded when the implant is arranged in the first state and a restoring force is generated that forces the implant to assume the second state when the implant is released from the expansion device.

9. The implant delivery system according to claim 8, wherein the connecting portion comprises a meandering shape.

10. The implant delivery system according to claim 1, wherein the connecting portion comprises a first part connected to the first end section and a second part connected to the second end section of the implant, wherein in the first state of the implant the first part and the second part are disconnected from one another, and wherein in the second state, the first part and the second part are connected to one another.

11. The implant according to claim 10, wherein the first and the second part are one of:
configured to be connected by at least one snap-in connection formed by the first and the second part in the second state of the implant,
configured to be connected by a magnetic force in the second state of the implant,
configured to be connected by engaging with one another, wherein the first part comprises at least one loop engaging with at least one hook of the second part in the second state of the implant.

12. The implant delivery system according to claim 5, wherein the catheter comprises a movable sheath configured to be advanced with respect to the outer sheath so as to move the first leg and the second leg towards one another and to thereby connect the first part to the second part of the connecting portion in the second state of the implant.

13. The implant delivery system according to claim 3, comprising a plurality of first end sections and a plurality of opposing second end sections, wherein the plurality of first end sections are rigidly connected to one another, and wherein the second end sections are rigidly connected to one another, and wherein the connecting portion is configured to connect the plurality of first and second end sections, wherein the connecting portion is an elongated flexible member connecting the plurality of first end sections to the plurality of second end sections such that a pulling force applied to the connecting portion pulls the plurality of first end sections and second end sections towards one another so as to arrange the implant in the second state.

14. The implant delivery system according to claim 1, wherein the implant delivery system further comprises an elongated guiding member arranged in a lumen of the catheter sheath, the guiding member comprising a first and a second sidewall opposing one another and being connected by a connecting member, wherein the first side wall comprises an elongated first slot, and wherein the second side wall comprises an elongated second slot, wherein the first and the second leg of the expansion device are configured to be slidably arranged in the lumen of the catheter sheath between the first sidewall and the second sidewall of the guiding member, and wherein the implant is configured to be arranged in the lumen of the catheter sheath so that the first connection arm engages into the first slot and into a first opening of the first leg of the expansion device and the second connection arm engages into the second slot and into a second opening of the second leg of the expansion device.

15. The implant delivery system according to claim 14, wherein the first and the second leg are configured to spread apart from one another when the first and the second legs are moved out of the lumen of the catheter sheath through an opening of the catheter sheath at a distal end of the catheter sheath to anchor the first and the second fixation arm in the inner wall (W) of the vessel (V) and to expand the contracting member of the implant, and wherein the first connection arm moves out of the first opening of the first leg and the second connection arm moves out of the second opening of the second leg of the expansion device, when the expansion device is retracted into the lumen of the catheter sheath.
